SEQUENCE LISTING (102 sequences)

SEQ ID NO: 1 - DNA, 23 bp, synthetic construct
  source 1..23: mol_type = other DNA
agccccctcagggtccagggggc

SEQ ID NO: 2 - DNA, 23 bp, synthetic construct
  source 1..23: mol_type = other DNA
aggccccctgctacgtgggggcc

SEQ ID NO: 3 - DNA, 23 bp, synthetic construct
  source 1..23: mol_type = other DNA
agggcccctgactgccagggccc

SEQ ID NO: 4 - DNA, 23 bp, synthetic construct
  source 1..23: mol_type = other DNA
aggggccctggcatgctggcccc

SEQ ID NO: 5 - DNA, 23 bp, synthetic construct
  source 1..23: mol_type = other DNA
agggggccaaggtccgagccccc

SEQ ID NO: 6 - DNA, 23 bp, synthetic construct
  source 1..23: mol_type = other DNA
aggggggcgacgtcggtcccccc

SEQ ID NO: 7 - DNA, 23 bp, synthetic construct
  source 1..23: mol_type = other DNA
agccgggcgacgtcggtcccggc

SEQ ID NO: 8 - DNA, 28 bp, synthetic construct
  source 1..28: mol_type = other DNA
catgctgaggtgtagacccatggattcc

SEQ ID NO: 9 - DNA, 25 bp, synthetic construct
  source 1..25: mol_type = other DNA
catgctgttaccaggccccctctcc

SEQ ID NO: 10 - DNA, 30 bp, synthetic construct
  source 1..30: mol_type = other DNA
catgctcatcccaaaatatgggctaacacc

SEQ ID NO: 11 - DNA, 26 bp, synthetic construct
  source 1..26: mol_type = other DNA
catgctcgatctcgaatcgaagcacc

SEQ ID NO: 12 - DNA, 27 bp, synthetic construct
  source 1..27: mol_type = other DNA
catggaaatgggtagacaaagcaggcc

SEQ ID NO: 13 - DNA, 25 bp, synthetic construct
  source 1..25: mol_type = other DNA
catgctgtctgcaacacgaagctcc

SEQ ID NO: 14 - DNA, 25 bp, synthetic construct
  source 1..25: mol_type = other DNA
catgggaaaaagagcttcgtgcagc

SEQ ID NO: 15 - DNA, 41 bp, synthetic construct
  source 1..41: mol_type = other DNA
gaatccatgggtctacacctcagccttgacatgctgagaac

SEQ ID NO: 16 - DNA, 44 bp, synthetic construct
  source 1..44: mol_type = other DNA
gaatccatgggtctacacctcagcaagggagtaaagttaatacc

SEQ ID NO: 17 - DNA, 43 bp, synthetic construct
  source 1..43: mol_type = other DNA
gaatccatgggtctacacctcagcataacctcatcgattgacg

SEQ ID NO: 18 - DNA, 42 bp, synthetic construct
  source 1..42: mol_type = other DNA
gaatccatgggtctacacctcagcttgacatactagaaactt

SEQ ID NO: 19 - DNA, 39 bp, synthetic construct
  source 1..39: mol_type = other DNA
gagagggggcctggtaacagcatagagccttccccttcg

SEQ ID NO: 20 - DNA, 39 bp, synthetic construct
  source 1..39: mol_type = other DNA
gagagggggcctggtaacagcatagagcttccccttcgg

SEQ ID NO: 21 - DNA, 37 bp, synthetic construct
  source 1..37: mol_type = other DNA
gagagggggcctggtaacagcagagctttcccttcgg

SEQ ID NO: 22 - DNA, 40 bp, synthetic construct
  source 1..40: mol_type = other DNA
gagagggggcctggtaacagcaaggtgttgtggttaataa

SEQ ID NO: 23 - DNA, 45 bp, synthetic construct
  source 1..45: mol_type = other DNA
gtgttagcccatattttgggatgagcctgttatcccaayacaaac

SEQ ID NO: 24 - DNA, 42 bp, synthetic construct
  source 1..42: mol_type = other DNA
gtgttagcccatattttgggatgagcactaccagcagatgcg

SEQ ID NO: 25 - DNA, 46 bp, synthetic construct
  source 1..46: mol_type = other DNA
gtgttagcccatattttgggatgagcattagtatcggaagaactca

SEQ ID NO: 26 - DNA, 49 bp, synthetic construct
  source 1..49: mol_type = other DNA
gtgttagcccatattttgggatgagcagtatgtattctttaacacgttg

SEQ ID NO: 27 - DNA, 36 bp, synthetic construct
  source 1..36: mol_type = other DNA
gtgcttcgattcgagatcgagccgctgcggctagac

SEQ ID NO: 28 - DNA, 35 bp, synthetic construct
  source 1..35: mol_type = other DNA
gtgcttcgattcgagatcgagccggtcgcgctggc

SEQ ID NO: 29 - DNA, 40 bp, synthetic construct
  source 1..40: mol_type = other DNA
gtgcttcgattcgagatcgagatttgggcgtggttaagga

SEQ ID NO: 30 - DNA, 34 bp, synthetic construct
  source 1..34: mol_type = other DNA
gtgcttcgattcgagatcaagagtgatgcgtctc

SEQ ID NO: 31 - DNA, 38 bp, synthetic construct
  source 1..38: mol_type = other DNA
gcctgctttgtctacccatttccagcagaggagcgagg

SEQ ID NO: 32 - DNA, 34 bp, synthetic construct
  source 1..34: mol_type = other DNA
gcctgctttgtctacccagagaatacgaaactcg

SEQ ID NO: 33 - DNA, 42 bp, synthetic construct
  source 1..42: mol_type = other DNA
gcctgctttgtctacccatttgaaccaatatacatcggaata

SEQ ID NO: 34 - DNA, 44 bp, synthetic construct
  source 1..44: mol_type = other DNA
gagcttcgtgttgcagacagcatgttaaagaagatggtatgtgg

SEQ ID NO: 35 - DNA, 40 bp, synthetic construct
  source 1..40: mol_type = other DNA
gagcttcgtgttgcagacagccttttataaagcacaaatc

SEQ ID NO: 36 - DNA, 43 bp, synthetic construct
  source 1..43: mol_type = other DNA
ctgcacgaagctctttttcccgcgacggatctacgtcacagcg

SEQ ID NO: 37 - DNA, 20 bp, synthetic construct
  source 1..20: mol_type = other DNA
gcaacgcgaagaaccttacc

SEQ ID NO: 38 - DNA, 19 bp, synthetic construct
  source 1..19: mol_type = other DNA
gttcccgaaggcaccaatc

SEQ ID NO: 39 - DNA, 21 bp, synthetic construct
  source 1..21: mol_type = other DNA
ggccttcgggttgtaaagtac

SEQ ID NO: 40 - DNA, 21 bp, synthetic construct
  source 1..21: mol_type = other DNA
tcttctgcgggtaacgtcaat

SEQ ID NO: 41 - DNA, 18 bp, synthetic construct
  source 1..18: mol_type = other DNA
agcctgatgcagccatgc

SEQ ID NO: 42 - DNA, 20 bp, synthetic construct
  source 1..20: mol_type = other DNA
ttagccggtgcttcttctgc

SEQ ID NO: 43 - DNA, 20 bp, synthetic construct
  source 1..20: mol_type = other DNA
gcaacgcgaagaaccttacc

SEQ ID NO: 44 - DNA, 19 bp, synthetic construct
  source 1..19: mol_type = other DNA
gttcccgaaggcaccaatc

SEQ ID NO: 45 - DNA, 20 bp, synthetic construct
  source 1..20: mol_type = other DNA
gcaacgcgaagaaccttacc

SEQ ID NO: 46 - DNA, 21 bp, synthetic construct
  source 1..21: mol_type = other DNA
catgcaccacctgtcactttg

SEQ ID NO: 47 - DNA, 20 bp, synthetic construct
  source 1..20: mol_type = other DNA
gcaacgcgaagaaccttacc

SEQ ID NO: 48 - DNA, 21 bp, synthetic construct
  source 1..21: mol_type = other DNA
catgcaccacctgtcactttg

SEQ ID NO: 49 - DNA, 24 bp, synthetic construct
  source 1..24: mol_type = other DNA
ggtggagcatgtggtttaattcga

SEQ ID NO: 50 - DNA, 21 bp, synthetic construct
  source 1..21: mol_type = other DNA
ctcacgacacgagctgacgac

SEQ ID NO: 51 - DNA, 19 bp, synthetic construct
  source 1..19: mol_type = other DNA
tcctacgggaggcagcagt

SEQ ID NO: 52 - DNA, 19 bp, synthetic construct
  source 1..19: mol_type = other DNA
ctgctggcacggagttagc

SEQ ID NO: 53 - DNA, 21 bp, synthetic construct
  source 1..21: mol_type = other DNA
tgcccaacagcaactrsaagt

SEQ ID NO: 54 - DNA, 23 bp, synthetic construct
  source 1..23: mol_type = other DNA
gaagccatttgttgtgatgtttg

SEQ ID NO: 55 - DNA, 25 bp, synthetic construct
  source 1..25: mol_type = other DNA
gagtttcttcgacaacgggtactac

SEQ ID NO: 56 - DNA, 17 bp, synthetic construct
  source 1..17: mol_type = other DNA
tcctccggcaccacatg

SEQ ID NO: 57 - DNA, 22 bp, synthetic construct
  source 1..22: mol_type = other DNA
tgtggcgtcattgtgtaacaaa

SEQ ID NO: 58 - DNA, 23 bp, synthetic construct
  source 1..23: mol_type = other DNA
attgtgttgtttcaccgatgttg

SEQ ID NO: 59 - DNA, 22 bp, synthetic construct
  source 1..22: mol_type = other DNA
catccattggttgatttttcca

SEQ ID NO: 60 - DNA, 23 bp, synthetic construct
  source 1..23: mol_type = other DNA
tggatcgacaaaaagaaaaagga

SEQ ID NO: 61 - DNA, 20 bp, synthetic construct
  source 1..20: mol_type = other DNA
ggcgcctaacaaggatgaca

SEQ ID NO: 62 - DNA, 18 bp, synthetic construct
  source 1..18: mol_type = other DNA
gacgcccaatccctcgag

SEQ ID NO: 63 - DNA, 22 bp, synthetic construct
  source 1..22: mol_type = other DNA
ctggatcaagcaggagatcaac

SEQ ID NO: 64 - DNA, 19 bp, synthetic construct
  source 1..19: mol_type = other DNA
cgcccatcttgtcctgatg

SEQ ID NO: 65 - DNA, 20 bp, synthetic construct
  source 1..20: mol_type = other DNA
cccaatagcttcatcgccct

SEQ ID NO: 66 - DNA, 24 bp, synthetic construct
  source 1..24: mol_type = other DNA
gtccatcccacttaaagacttggt

SEQ ID NO: 67 - DNA, 23 bp, synthetic construct
  source 1..23: mol_type = other DNA
gcaaaactggttgttccargtca

SEQ ID NO: 68 - DNA, 22 bp, synthetic construct
  source 1..22: mol_type = other DNA
aaycctttaaccgcctgctcta

SEQ ID NO: 69 - DNA, 17 bp, synthetic construct
  source 1..17: mol_type = other DNA
ccgttcccgcagacctt

SEQ ID NO: 70 - DNA, 22 bp, synthetic construct
  source 1..22: mol_type = other DNA
ttttttgccgtttcctgtatcc

SEQ ID NO: 71 - DNA, 23 bp, synthetic construct
  source 1..23: mol_type = other DNA
catggtctgcttgtcatgaaaga

SEQ ID NO: 72 - DNA, 20 bp, synthetic construct
  source 1..20: mol_type = other DNA
gggaaagccacgtcaatacg

SEQ ID NO: 73 - DNA, 23 bp, synthetic construct
  source 1..23: mol_type = other DNA
gcagagattgccaacaacattga

SEQ ID NO: 74 - DNA, 19 bp, synthetic construct
  source 1..19: mol_type = other DNA
gcatgttttccaaacgcca

SEQ ID NO: 75 - DNA, 24 bp, synthetic construct
  source 1..24: mol_type = other DNA
aactacggtaacattgatcgcaac

SEQ ID NO: 76 - DNA, 23 bp, synthetic construct
  source 1..23: mol_type = other DNA
gcattcctggaataatgacgcta

SEQ ID NO: 77 - DNA, 21 bp, synthetic construct
  source 1..21: mol_type = other DNA
cggtgaaaatatcccgagtga

SEQ ID NO: 78 - DNA, 25 bp, synthetic construct
  source 1..25: mol_type = other DNA
ggccatatcctgaatctgctaataa

SEQ ID NO: 79 - DNA, 21 bp, synthetic construct
  source 1..21: mol_type = other DNA
gcttcttgtggagctcgacaa

SEQ ID NO: 80 - DNA, 21 bp, synthetic construct
  source 1..21: mol_type = other DNA
ccgtcagcaacttcgttttca

SEQ ID NO: 81 - DNA, 18 bp, synthetic construct
  source 1..18: mol_type = other DNA
ccttgacatgctgagaac

SEQ ID NO: 82 - DNA, 20 bp, synthetic construct
  source 1..20: mol_type = other DNA
aagggagtaaagttaatacc

SEQ ID NO: 83 - DNA, 18 bp, synthetic construct
  source 1..18: mol_type = other DNA
taacctcatcgattgacg

SEQ ID NO: 84 - DNA, 20 bp, synthetic construct
  source 1..20: mol_type = other DNA
ccttgacatactagaaactt

SEQ ID NO: 85 - DNA, 17 bp, synthetic construct
  source 1..17: mol_type = other DNA
tagagccttccccttcg

SEQ ID NO: 86 - DNA, 18 bp, synthetic construct
  source 1..18: mol_type = other DNA
atagagcttccccttcgg

SEQ ID NO: 87 - DNA, 16 bp, synthetic construct
  source 1..16: mol_type = other DNA
agagctttcccttcgg

SEQ ID NO: 88 - DNA, 19 bp, synthetic construct
  source 1..19: mol_type = other DNA
aaggtgttgtggttaataa

SEQ ID NO: 89 - DNA, 20 bp, synthetic construct
  source 1..20: mol_type = other DNA
cctgttatcccaayacaaac

SEQ ID NO: 90 - DNA, 16 bp, synthetic construct
  source 1..16: mol_type = other DNA
actaccagcagatgcg

SEQ ID NO: 91 - DNA, 19 bp, synthetic construct
  source 1..19: mol_type = other DNA
ttagtatcggaagaactca

SEQ ID NO: 92 - DNA, 24 bp, synthetic construct
  source 1..24: mol_type = other DNA
cagtatgtattctttaacacgttg

SEQ ID NO: 93 - DNA, 16 bp, synthetic construct
  source 1..16: mol_type = other DNA
gccgctgcggctagac

SEQ ID NO: 94 - DNA, 15 bp, synthetic construct
  source 1..15: mol_type = other DNA
gccggtcgcgctggc

SEQ ID NO: 95 - DNA, 20 bp, synthetic construct
  source 1..20: mol_type = other DNA
gatttgggcgtggttaagga

SEQ ID NO: 96 - DNA, 17 bp, synthetic construct
  source 1..17: mol_type = other DNA
caagagtgatgcgtctc

SEQ ID NO: 97 - DNA, 16 bp, synthetic construct
  source 1..16: mol_type = other DNA
cagcagaggagcgagg

SEQ ID NO: 98 - DNA, 17 bp, synthetic construct
  source 1..17: mol_type = other DNA
agagaatacgaaactcg

SEQ ID NO: 99 - DNA, 22 bp, synthetic construct
  source 1..22: mol_type = other DNA
tgaaccaatatacatcggaata

SEQ ID NO: 100 - DNA, 22 bp, synthetic construct
  source 1..22: mol_type = other DNA
tgttaaagaagatggtatgtgg

SEQ ID NO: 101 - DNA, 20 bp, synthetic construct
  source 1..20: mol_type = other DNA
ccttttataaagcacaaatc

SEQ ID NO: 102 - DNA, 23 bp, synthetic construct
  source 1..23: mol_type = other DNA
cgcgacggatctacgtcacagcg

(19) United States
(12) Patent Application Publication
(10) Pub. No.: US 2024/0084405 A1
(43) Pub. Date: Mar. 14, 2024

(54) UNIVERSAL PROBE, PRIMER-PROBE SET AND KIT

(71) Applicant: PILOT GENE TECHNOLOGY (HANGZHOU) CO., LTD., Hangzhou (CN)

(72) Inventors: Jiang XIA, Hangzhou (CN); Hao Yu, Hangzhou (CN)

(21) Appl. No.: 18/326,300

(22) Filed: May 31, 2023

Related U.S. Application Data
(63) Continuation of application No. PCT/CN23/92551, filed on May 6, 2023.

(30) Foreign Application Priority Data
Aug. 31, 2022 (CN) 202211052186.6

(51) Int. Cl. C12Q 1/689; C12Q 1/6895

(52) U.S. Cl. CPC C12Q 1/689; C12Q 1/6895; C12Q 1/6844

(57) ABSTRACT

The present application relates to a field of a biological detection technology, in particular, relates to a universal probe, a primer-probe set and a kit. The present application provides a universal probe, in which, from 5′ end to 3′ end, the universal probe sequentially comprises: quenching group-fragment A -fluorophore-fragment B-C3; the fragment A has a nucleotide sequence selected from a group consisting of SEQ ID NO:1-7; and the fragment B has a nucleotide sequence selected from a group consisting of SEQ ID NO:8-14. The present application further provide a primer-probe set, an application and the kit.

### CROSS-REFERENCE TO RELATED APPLICATION

This application is a continuation of PCT application serial no. PCT/CN2023/092551, filed on May 6, 2023, which claims the priority and benefit of Chinese patent application serial no. 202211052186.6, filed on Aug. 31, 2022. The entireties of PCT application serial no. PCT/CN2023/092551 and Chinese patent application serial no. 202211052186.6 are hereby incorporated by reference herein and made a part of this specification.

### REFERENCE TO AN ELECTRONIC SEQUENCE LISTING

The contents of the electronic sequence listing (SequenceListing.xml; Size: 91,085 bytes; and Date of Creation: May 31, 2023) is herein incorporated by reference.

### TECHNICAL FIELD

The present application relates to a field of a biological detection technology, in particular, relates to a universal probe, a primer-probe set and a kit.

### BACKGROUND ART

A pathogenic microorganism is a microorganism that can cause an infectious disease or even a communicable disease after invading a human body. Common pathogenic microorganisms include prion, bacterium, mycoplasma, chlamydia, rickettsia, fungi, spirochete and virus. After repetitive exposure to some chemotherapeutic drugs, a reactivity of these pathogenic microorganisms continues to decrease, so that finally the pathogenic microorganisms can resist the drug without being killed or inhibited, and thus develop a tolerance to drugs, that is, a drug tolerance or drug resistance. A resistant gene is a nucleotide sequence encoding a drug resistance property, and is located on a chromosome of the bacterium or a plasmid outside the chromosomal. If the pathogenic microorganisms develop the drug resistance, an efficacy is commonly reduced or completely lost, which directly affect a therapeutic effect against the disease. Thus, establishing a systematic, efficient, and fast detection method for identifying a nucleic acid of a plurality of pathogenic microorganisms and resistant genes can effectively promote the development of the medical industry.

At present, a conventional detection method of nucleic acids of the pathogenic microorganisms and resistant genes mainly adopts a real-time quantitative PCR (qPCR), including a fluorescent dye method (SYBR Green I method is a representative) and a fluorescent probe method (TaqMan method is the representative). The SYBR Green I method is widely used due to advantages such as convenient use and low price, but a biggest disadvantage thereof is a lack of a specificity, that is, a dye can be combined with any dsDNA, which affects an accuracy of a quantitative result. Different from the fluorescent dye method, the TaqMan method detection involves an addition of a fluorescent probe, which works through fluorescence resonance energy transfer (FRET), and has a high specificity and suitability for multiplex qPCR. However, a traditional TaqMan probe technology usually requires one probe for each of the targets. However, when detecting multiple targets in one fluorescence channel, if there are too many probes labeled with a same fluorescence, it will lead to a high background signal and decreased contrast, resulting in difficult signal interpretation difficult and limited number of targets that can be detected.

### SUMMARY

In view of this, a technical problem to be solved by the present application is to provide a primer-probe set and a kit containing the primer-probe for a digital PCR detection of a plurality of pathogenic microorganisms and resistant genes. The present application provides a digital PCR platform based on a 7-color fluorescence channel and in combination with multiplex digital PCR technology to achieve a coverage of multiple bacterial strains or drug resistance sites for each species.

The present application provides a universal probe structure, from 5′ end to 3′ end, the universal probe sequentially including: quenching group-fragment A-fluorophore-fragment B-C3.

In some embodiments, the fragment A has a nucleotide sequence selected from a group consisting of SEQ ID NO:1-7.

the fragment B has a nucleotide sequence selected from a group consisting of SEQ ID NO:8-14.

In some embodiments, from 5′ end to 3′ end, the universal probe sequentially includes one selected from a group consisting of:
quenching group-fragment A having a nucleotide sequence of SEQ ID NO:1-fluorophore-fragment B-C3 having a nucleotide sequence of SEQ ID NO:8;
quenching group-fragment A having a nucleotide sequence of SEQ ID NO:2-fluorophore-fragment B-C3 having a nucleotide sequence of SEQ ID NO:9;
quenching group-fragment A having a nucleotide sequence of SEQ ID NO:3-fluorophore-fragment B-C3 having a nucleotide sequence of SEQ ID NO:10;
quenching group-fragment A having a nucleotide sequence of SEQ ID NO:4-fluorophore-fragment B-C3 having a nucleotide sequence of SEQ ID NO:11;
quenching group-fragment A having a nucleotide sequence of SEQ ID NO:5-fluorophore-fragment B-C3 having a nucleotide sequence of SEQ ID NO:12;
quenching group-fragment A having a nucleotide sequence of SEQ ID NO:6-fluorophore-fragment B-C3 having a nucleotide sequence of SEQ ID NO:13; and
quenching group-fragment A having a nucleotide sequence of SEQ ID NO:7-fluorophore-fragment B-C3 having a nucleotide sequence of SEQ ID NO:14.

The fluorophore of the universal probe described in the present application is selected from a group consisting of FAM, VIC, ROX, CY5, A425, CY5.5 and CY7; and the quenching group is selected from a group consisting of BHQ1, BHQ2 and BHQ3.

In some embodiments, the fluorophore A425 of the universal probe is fluorophore ATT0425.

In some embodiments, from 5′ end to 3′ end, the universal probe sequentially includes one selected from a group consisting of:
BHQ1-fragment A having a nucleotide sequence of SEQ ID NO:1-dT-FAM-fragment B-C3 having a nucleotide sequence of SEQ ID NO:8;
BHQ1-fragment A having a nucleotide sequence of SEQ ID NO:2-dT-VIC-fragment B-C3 having a nucleotide sequence of SEQ ID NO:9;
BHQ2-fragment A having a nucleotide sequence of SEQ ID NO:3-dT-ROX-fragment B-C3 having a nucleotide sequence of SEQ ID NO:10;
BHQ2-fragment A having a nucleotide sequence of SEQ ID NO:4-dT-CY5-fragment B-C3 having a nucleotide sequence of SEQ ID NO:11;
BHQ1-fragment A having a nucleotide sequence of SEQ ID NO:5-dT-A425-fragment B-C3 having a nucleotide sequence of SEQ ID NO:12;
BHQ3-fragment A having a nucleotide sequence of SEQ ID NO:6-dT-CY5.5 -fragment B-C3 having a nucleotide sequence of SEQ ID NO:13; and
BHQ3-fragment A having a nucleotide sequence of SEQ ID NO:7-dT-CY7-fragment B-C3 having a nucleotide sequence of SEQ ID NO:14.

In the present application, the universal probe is in cooperation with a single-modified fluorescence-free probe. In particular, the single-modified fluorescence-free probe is specifically combined with an amplification product, and the universal probe is hybridized with a hydrolyzed fragment of the single-modified fluorescence-free probe to generate a fluorescent signal for a detection purpose.

The present application further provides a primer-probe set for detecting one selected from a group consisting of the pathogenic microorganism and the resistant gene, including the universal probe described in the present application.

The primer-probe set provided in the present application further includes a specific primer pair targeting the pathogenic microorganism and the resistant gene.

In particular, the pathogenic microorganism includes one selected from a group consisting of Pseudomonas aeruginosa, Escherichia coli, Klebsiella pneumoniae, Acinetobacter baumannii, Staphylococcus aureus, Enterococcus faecium, Enterococcus faecalis, Enterobacter cloacae, Candida albicans, Candida glabrata, Candida parapsilosis and Candida tropicalis;
the resistant gene includes one selected from a group consisting of carbapenem resistant KPC gene, carbapenem resistant NDM gene, carbapenem resistant OXA48 gene, carbapenem resistant IMP gene, vancomycin resistant vanA gene, vancomycin resistant vanB gene, vancomycin resistant vanM gene, methicillin resistant mecA gene and methicillin resistant mecC gene.

Specifically, a primer pair targeting the Pseudomonas aeruginosa has a nucleotide sequence selected from a group consisting of SEQ ID NO:37-38;
a primer pair targeting the Escherichia coli has a nucleotide sequence selected from a group consisting of SEQ ID NO:39-40;
a primer pair targeting the Klebsiella pneumoniae has a nucleotide sequence selected from a group consisting of SEQ ID NO:41-42;
a primer pair targeting the Acinetobacter baumannii has a nucleotide sequence selected from a group consisting of SEQ ID NO:43-44;
a primer pair targeting the Staphylococcus aureus has a nucleotide sequence selected from a group consisting of SEQ ID NO:45-46;
a primer pair targeting the Enterococcus faecium has a nucleotide sequence selected from a group consisting of SEQ ID NO:47-48;
a primer pair targeting the Enterococcus faecalis has a nucleotide sequence selected from a group consisting of SEQ ID NO:49-50;
a primer pair targeting the Enterobacter cloacae has a nucleotide sequence selected from a group consisting of SEQ ID NO:51-52;
a primer pair targeting the Candida albicans has a nucleotide sequence selected from a group consisting of SEQ ID NO:53-54;
a primer pair targeting the Candida glabrata has a nucleotide sequence selected from a group consisting of SEQ ID NO:55-56;
a primer pair targeting the Candida parapsilosis has a nucleotide sequence selected from a group consisting of SEQ ID NO:57-58;
a primer pair targeting the Candida tropicalis has a nucleotide sequence selected from a group consisting of SEQ ID NO:59-60;
a primer pair targeting the carbapenem resistant KPC gene has a nucleotide sequence selected from a group consisting of SEQ ID NO:61-62;
a primer pair targeting the carbapenem resistant NDM gene has a nucleotide sequence selected from a group consisting of SEQ ID NO:63-64;
a primer pair targeting the carbapenem resistant OXA48 gene has a nucleotide sequence selected from a group consisting of SEQ ID NO:65-66;
a primer pair targeting the carbapenem resistant IMP gene has a nucleotide sequence selected from a group consisting of SEQ ID NO:67-68;
a primer pair targeting the vancomycin resistant vanA gene has a nucleotide sequence selected from a group consisting of SEQ ID NO:69-70;
a primer pair targeting the vancomycin resistant vanB gene has a nucleotide sequence selected from a group consisting of SEQ ID NO:71-72;
a primer pair targeting the vancomycin resistant vanM gene has a nucleotide sequence selected from a group consisting of SEQ ID NO:73-74;
a primer pair targeting the methicillin resistant mecA gene has a nucleotide sequence selected from a group consisting of SEQ ID NO:75-76; and,
a primer pair targeting the methicillin resistant mecC gene has a nucleotide sequence selected from a group consisting of SEQ ID NO:77-78.

The primer-probe set provided in the present application further includes the single-modified fluorescence-free probe, a nucleotide sequence of the single-modified fluorescence-free probe is reversely complementary to a nucleotide sequence of the fragment B in the universal probe, and 3′ end of the single-modified fluorescence-free probe is labeled with the quenching group. In particular, the quenching group is selected from a group consisting of MGB, BHQ1, BHQ2 and BHQ3.

Specifically, a single-modified fluorescence-free probe targeting the Pseudomonas aeruginosa has a nucleotide sequence as shown in SEQ ID NO:15, and 3′ end is labeled with the MGB quenching group;
a single-modified fluorescence-free probe targeting the Escherichia coli has a nucleotide sequence as shown in SEQ ID NO:16, and 3′ end is labeled with the MGB quenching group;
a single-modified fluorescence-free probe targeting the Klebsiella pneumoniae has a nucleotide sequence as shown in SEQ ID NO:17, and 3′ end is labeled with the MGB quenching group;
a single-modified fluorescence-free probe targeting the Acinetobacter baumannii has a nucleotide sequence as shown in SEQ ID NO:18, and 3′ end is labeled with the MGB quenching group;
a single-modified fluorescence-free probe targeting the Staphylococcus aureus has a nucleotide sequence as shown in SEQ ID NO:19, and 3′ end is labeled with the MGB quenching group;
a single-modified fluorescence-free probe targeting the Enterococcus faecium has a nucleotide sequence as shown in SEQ ID NO:20, and 3′ end is labeled with the MGB quenching group;
a single-modified fluorescence-free probe targeting the Enterococcus faecalis has a nucleotide sequence as shown in SEQ ID NO:21, and 3′ end is labeled with the MGB quenching group;
a single-modified fluorescence-free probe targeting the Enterobacter cloacae has a nucleotide sequence as shown in SEQ ID NO:22, and 3′ end is labeled with the MGB quenching group;
a single-modified fluorescence-free probe targeting the Candida albicans has a nucleotide sequence as shown in SEQ ID NO:23, and 3′ end is labeled with the MGB quenching group;
a single-modified fluorescence-free probe targeting the Candida glabrata has a nucleotide sequence as shown in SEQ ID NO:24, and 3′ end is labeled with the MGB quenching group;
a single-modified fluorescence-free probe targeting the Candida parapsilosis has a nucleotide sequence as shown in SEQ ID NO:25, and 3′ end is labeled with the MGB quenching group;
a single-modified fluorescence-free probe targeting the Candida tropicalis has a nucleotide sequence as shown in SEQ ID NO:26, and 3′ end is labeled with the MGB quenching group;
a single-modified fluorescence-free probe targeting the carbapenem resistant KPC gene has a nucleotide sequence as shown in SEQ ID NO:27, and 3′ end is labeled with the MGB quenching group;
a single-modified fluorescence-free probe targeting the carbapenem resistant NDM gene has a nucleotide sequence as shown in SEQ ID NO:28, and 3′ end is labeled with the BHQ2 quenching group;
a single-modified fluorescence-free probe targeting the carbapenem resistant OXA48 gene has a nucleotide sequence as shown in SEQ ID NO:29, and 3′ end is labeled with the MGB quenching group;
a single-modified fluorescence-free probe targeting the carbapenem resistant IMP gene has a nucleotide sequence as shown in SEQ ID NO:30, and 3′ end is labeled with the MGB quenching group;
a single-modified fluorescence-free probe targeting the vancomycin resistant vanA gene has a nucleotide sequence as shown in SEQ ID NO:31, and 3′ end is labeled with the BHQ1 quenching group;
a single-modified fluorescence-free probe targeting the vancomycin resistant vanB gene has a nucleotide sequence as shown in SEQ ID NO:32, and 3′ end is labeled with the BHQ1 quenching group;
a single-modified fluorescence-free probe targeting the vancomycin resistant vanM gene has a nucleotide sequence as shown in SEQ ID NO:33, and 3′ end is labeled with the BHQ1 quenching group;
a single-modified fluorescence-free probe targeting the methicillin resistant mecA gene has a nucleotide sequence as shown in SEQ ID NO:34, and 3′ end is labeled with the BHQ2 quenching group;
a single-modified fluorescence-free probe targeting the methicillin resistant mecC gene has a nucleotide sequence as shown in SEQ ID NO:35, and 3′ end is labeled with the MGB quenching group.

The present application further provides an application of the universal probe or the primer-probe set in a preparation of digital PCR detection reagents for a plurality of pathogenic microorganisms and resistant genes.

The present application further provides a kit for detecting one selected from a group consisting of a plurality of pathogenic microorganisms and resistant genes, including one selected from a group consisting of the universal probe or the primer-probe set described in the present application.

The kit provided in the present application further includes a combination of a primer and a probe for an internal control gene, having the following sequences: IC-UP7: ctgcacgaagctctttttcccgcgacggatctacgtcacagcg (SEQ ID NO:36); IC-P: cgcgacggatctacgtcacagcg (SEQ ID NO:102); IC-F: gcttcttgtggagctcgacaa (SEQ ID NO:79); IC-R: ccgtcagcaacttcgttttca (SEQ ID NO:80).

The present application further provides a multiplex digital PCR detection method for a plurality of pathogenic microorganisms and resistant genes, utilizing the primer-probe set or the kit of the present application for detection.

The detection method based on a multiplex digital PCR platform includes the following steps:
(1) a nucleic acid of a sample to be tested is extracted;
(2) multiplex digital PCR amplification system is configured;
(3) an amplification program is run: 95 ° C. 5 min, (95 ° C. 15 s, 60 ° C. 1 min) x 40;
(4) a result is output.

By using the primer-probe of the present application, a joint detection of 12 pathogenic microorganisms and 9 resistant genes can be achieved. In particular, the pathogenic microorganisms includes one selected from a group consisting of Pseudomonas aeruginosa, Escherichia coli, Klebsiella pneumoniae, Acinetobacter baumannii, Staphylococcus aureus, Enterococcus faecium, Enterococcus faecalis, Enterobacter cloacae, Candida albicans, Candida glabrata, Candida parapsilosis and Candida tropicalis; and the resistant genes include one selected from a group consisting of carbapenem resistant KPC gene, carbapenem resistant NDM gene, carbapenem resistant OXA48 gene, carbapenem resistant IMP gene, vancomycin resistant vanA gene, vancomycin resistant vanB gene, vancomycin resistant vanM gene, methicillin resistant mecA gene and methicillin resistant mecC gene.

For conservative regions of 12 pathogenic microorganisms and 9 resistant genes, the present application specifically designs 42 primers, 7 universal probes containing different fluorophores and 21 specific single-modified fluorescence-free probes in total. The universal probe is cooperated with a single-modified probe to generate the fluorescent signal, which can be used to detect whether the sample contains Pseudomonas aeruginosa, Escherichia coli, Klebsiella pneumoniae, Acinetobacter baumannii, Staphylococcus aureus, Enterococcus faecium, Enterococcus faecalis, Enterobacter cloacae, Candida albicans, Candida glabrata, Candida parapsilosis or Candida tropicalis in the sample, and detect whether the sample contains a site of carbapenem resistant KPC gene, carbapenem resistant NDM gene, carbapenem resistant OXA48 gene, carbapenem resistant IMP gene, vancomycin resistant vanA gene, vancomycin resistant vanB gene, vancomycin resistant vanM gene, methicillin resistant mecA gene or methicillin resistant mecC gene.

The present application adopts a single amplification system with a conventional double-modified probe and a multiplex amplification system with a cooperation of the universal probe and the single-modified probe, and the detection results of the two systems are consistent with each other, which ensures a feasibility of designing the universal probe containing different fluorophores for a target gene and combining them with the specific single-modified fluorescence-free probe to conduct the multiplex digital PCR.

### DETAILED DESCRIPTION

The present application provides a primer-probe set and a kit thereof for a digital PCR detection of a plurality of pathogenic microorganisms and resistant genes, and those skilled in the art can appropriately improve a process parameter by learning from a content of the present application. It should be noted that, all similar replacements and modifications are apparent to those skilled in the art and are considered to fall within a scope of the present invention. A method and an application of the present application have been described through a preferred embodiment, and it is obvious that a relevant personnel can make modifications or appropriate changes and combinations of the method and application without departing from the content, spirit and scope of the present application to achieve and apply a technology of the present application.

Test materials used in the present application are all common commercially available and can be purchased in the market.

The present application is further described below in combination with examples.

### EXAMPLE 1

### Design of the Primer-Probe Set of the Present Application

A sequence of the primer-probe set involved in this example is shown in Table 1.

In particular, UP1-UP7 represents 7 universal probes with different colors. F represents an upstream primer, R represents a downstream primer, and P represents the probe. For example, TL-F represents an upstream primer of the Pseudomonas aeruginosa, TL-R represents a downstream primer of the Pseudomonas aeruginosa, TL-P represents a conventional double-modified probe of the Pseudomonas aeruginosa, and TL-UP1 represents the specific single-modified fluorescence-free probe of the Pseudomonas aeruginosa.

TABLE 1

| | | | 5′ | 3′ |
|---|---|---|---|---|
| | | | modi- | modi- |
| Name | Number | Sequence | fication | fication |
| universal probe | fragment A | Agccccctcagggtccagggggc (SEQ ID NO: 1) | BHQ1 | |
| universal probe | fragment A | aggccccctgctacgtgggggcc (SEQ ID NO: 2) | BHQ1 | |
| universal probe | fragment A | agggcccctgactgccagggccc (SEQ ID NO: 3) | BHQ2 | |
| universal probe | fragment A | aggggccctggcatgctggcccc (SEQ ID NO: 4) | BHQ2 | |
| universal probe | fragment A | agggggccaaggtccgagccccc (SEQ ID NO: 5) | BHQ1 | |
| universal probe | fragment A | aggggggcgacgtcggtcccccc (SEQ ID NO: 6) | BHQ3 | |
| universal probe | fragment A | agccgggcgacgtcggtcccggc (SEQ ID NO: 7) | BHQ3 | |
| universal probe | fragment B | catgctgaggtgtagacccatggattcc (SEQ ID NO: 8) | | C3 |
| universal probe | fragment B | catgctgttaccaggccccctctcc (SEQ ID NO: 9) | | C3 |
| universal probe | fragment B | catgctcatcccaaaatatgggctaacacc (SEQ ID | | C3 |
| | | NO: 10) | | |
| universal probe | fragment B | catgctcgatctcgaatcgaagcacc (SEQ ID NO: 11) | | C3 |
| universal probe | fragment B | catggaaatgggtagacaaagcaggcc (SEQ ID | | C3 |
| | | NO: 12) | | |
| universal probe | fragment B | catgctgtctgcaacacgaagctcc (SEQ ID NO: 13) | | C3 |
| universal probe | fragment B | catgggaaaaagagcttcgtgcagc (SEQ ID NO: 14) | | C3 |
| pseudomonas | TL-UP1 | gaatccatgggtctacacctcagccttgacatgctgagaac | | MGB |
| aeruginosa | | (SEQ ID NO: 15) | | |
| escherichia | DC-UP1 | gaatccatgggtctacacctcagcaagggagtaaagttaatac | | MGB |
| coli | | c (SEQ ID NO: 16) | | |
| klebsiella | FK-UP1 | gaatccatgggtctacacctcagcataacctcatcgattgacg | | MGB |
| pneumoniae | | (SEQ ID NO: 17) | | |
| acinetobacter | BM-UP1 | gaatccatgggtctacacctcagcttgacatactagaaactt | | MGB |
| baumannii | | (SEQ ID NO: 18) | | |
| staphylococcus | JP-UP2 | gagagggggcctggtaacagcatagagccttccccttcg | | MGB |
| aureus | | (SEQ ID NO: 19) | | |
| enterococcus | SC-UP2 | gagagggggcctggtaacagcatagagcttccccttcgg | | MGB |
| faecium | | (SEQ ID NO: 20) | | |
| enterococcus | FC-UP2 | gagagggggcctggtaacagcagagctttcccttcgg | | MGB |
| faecalis | | (SEQ ID NO: 21) | | |
| enterobacter | YG-UP2 | gagagggggcctggtaacagcaaggtgttgtggttaataa | | MGB |
| cloacae | | (SEQ ID NO: 22) | | |
| candida | BS-UP3 | gtgttagcccatattttgggatgagcctgttatcccaayaca | | MGB |
| albicans | | aac (SEQ ID NO: 23) | | |
| candida | GH-UP3 | gtgttagcccatattttgggatgagcactaccagcagatgcg | | MGB |
| glabrata | | (SEQ ID NO: 24) | | |
| candida | JPH-UP3 | gtgttagcccatattttgggatgagcattagtatcggaagaact | | MGB |
| parapsilosis | | ca (SEQ ID NO: 25) | | |
| candida | RD-UP3 | gtgttagcccatattttgggatgagcagtatgtattctttaac | | MGB |
| parapsilosis | | acgttg (SEQ ID NO: 26) | | |
| carbapenem | KPC-UP4 | gtgcttcgattcgagatcgagccgctgcggctagac (SEQ | | MGB |
| resistant KPC | | ID NO: 27) | | |
| carbapenem | NDM-UP4 | gtgcttcgattcgagatcgagccggtcgcgctggc (SEQ | | BHQ2 |
| resistant NDM | | ID NO: 28) | | |
| carbapenem | OXA48-UP4 | gtgcttcgattcgagatcgagatttgggcgtggttaagga | | MGB |
| resistant | | (SEQ ID NO: 29) | | |
| OXA48 | | | | |
| carbapenem | IMP-UP4 | gtgcttcgattcgagatcaagagtgatgcgtctc (SEQ ID | | MGB |
| resistant IMP | | NO: 30) | | |
| vancomycin | vanA-UP5 | gcctgctttgtctacccatttccagcagaggagcgagg | | BHQ1 |
| resistant vanA | | (SEQ ID NO: 31) | | |
| vancomycin | VANB-UP5 | gcctgctttgtctacccagagaatacgaaactcg (SEQ ID | | BHQ1 |
| resistant vanB | | NO: 32) | | |
| vancomycin | VANM-UP5 | gcctgctttgtctacccatttgaaccaatatacatcggaata | | BHQ1 |
| resistant vanM | | (SEQ ID NO: 33) | | |
| methicillin | MECA-UP6 | gagcttcgtgttgcagacagcatgttaaagaagatggtatgtg | | BHQ2 |
| resistant mecA | | g (SEQ ID NO: 34) | | |
| methicillin | mecC-UP6 | gagcttcgtgttgcagacagccttttataaagcacaaatc | | MGB |
| resistant mecC | | (SEQ ID NO: 35) | | |
| exogenous | IC-UP7 | ctgcacgaagctctttttcccgcgacggatctacgtcacagcg | | BHQ3 |
| internal control | | (SEQ ID NO: 36) | | |
| pseudomonas | TL-F | gcaacgegaagaaccttacc (SEQ ID NO: 37) | | |
| aeruginosa | | | | |
| pseudomonas | TL-R | gttcccgaaggcaccaatc (SEQ ID NO: 38) | | |
| aeruginosa | | | | |
| escherichia | DC-F | ggccttcgggttgtaaagtac (SEQ ID NO: 39) | | |
| coli | | | | |
| escherichia | DC-R | tcttctgcgggtaacgtcaat (SEQ ID NO: 40) | | |
| coli | | | | |
| klebsiella | FK-F | agcctgatgcagccatgc (SEQ ID NO: 41) | | |
| pneumoniae | | | | |
| klebsiella | FK-R | ttagccggtgcttcttctgc (SEQ ID NO: 42) | | |
| pneumoniae | | | | |
| acinetobacter | BM-F | gcaacgcgaagaaccttacc (SEQ ID NO: 43) | | |
| baumannii | | | | |
| acinetobacter | BM-R | gttcccgaaggcaccaatc (SEQ ID NO: 44) | | |
| baumannii | | | | |
| staphylococcus | JP-F | gcaacgcgaagaaccttacc (SEQ ID NO: 45) | | |
| aureus | | | | |
| staphylococcus | JP-R | catgcaccacctgtcactttg (SEQ ID NO: 46) | | |
| aureus | | | | |
| enterococcus | SC-F | gcaacgegaagaaccttacc (SEQ ID NO: 47) | | |
| faecium | | | | |
| enterococcus | SC-R | catgcaccacctgtcactttg (SEQ ID NO: 48) | | |
| faecium | | | | |
| enterococcus | FC-F | ggtggagcatgtggtttaattcga (SEQ ID NO: 49) | | |
| faecalis | | | | |
| enterococcus | FC-R | ctcacgacacgagctgacgac (SEQ ID NO: 50) | | |
| faecalis | | | | |
| enterobacter | YG-F | tcctacgggaggcagcagt (SEQ ID NO: 51) | | |
| cloacae | | | | |
| enterobacter | YG-R | ctgctggcacggagttagc (SEQ ID NO: 52) | | |
| cloacae | | | | |
| candida | BS-F | tgcccaacagcaactrsaagt (SEQ ID NO: 53) | | |
| albicans | | | | |
| candida | BS-R | gaagccatttgttgtgatgtttg (SEQ ID NO: 54) | | |
| albicans | | | | |
| candida | GH-F | gagtttcttcgacaacgggtactac (SEQ ID NO: 55) | | |
| glabrata | | | | |
| candida | GH-R | tcctccggcaccacatg (SEQ ID NO: 56) | | |
| glabrata | | | | |
| candida | JPH-F | tgtggcgtcattgtgtaacaaa (SEQ ID NO: 57) | | |
| parapsilosis | | | | |
| candida | JPH-R | attgtgttgtttcaccgatgttg (SEQ ID NO: 58) | | |
| parapsilosis | | | | |
| candida | RD-F | catccattggttgatttttcca (SEQ ID NO: 59) | | |
| tropicalis | | | | |
| candida | RD-R | tggatcgacaaaaagaaaaagga (SEQ ID NO: 60) | | |
| tropicalis | | | | |
| carbapenem | KPC-F | ggcgcctaacaaggatgaca (SEQ ID NO: 61) | | |
| resistant KPC | | | | |
| carbapenem | KPC-R | gacgcccaatccctcgag (SEQ ID NO: 62) | | |
| resistant KPC | | | | |
| carbapenem | NDM-F | ctggatcaagcaggagatcaac (SEQ ID NO: 63) | | |
| resistant NDM | | | | |
| carbapenem | NDM-R | cgcccatcttgtcctgatg (SEQ ID NO: 64) | | |
| resistant NDM | | | | |
| carbapenem | OXA48-F | cccaatagcttcatcgccct (SEQ ID NO: 65) | | |
| resistant | | | | |
| OXA48 | | | | |
| carbapenem | OXA48-R | gtccatcccacttaaagacttggt (SEQ ID NO: 66) | | |
| resistant | | | | |
| OXA48 | | | | |
| carbapenem | IMP-F | gcaaaactggttgttccargtca (SEQ ID NO: 67) | | |
| resistant IMP | | | | |
| carbapenem | IMP-R | aaycctttaaccgcctgctcta (SEQ ID NO: 68) | | |
| resistant IMP | | | | |
| vancomycin | vanA-F | ccgttcccgcagacctt (SEQ ID NO: 69) | | |
| resistant vanA | | | | |
| vancomycin | vanA-R | ttttttgccgtttcctgtatcc (SEQ ID NO: 70) | | |
| resistant vanA | | | | |
| vancomycin | vanB-F | catggtctgcttgtcatgaaaga (SEQ ID NO: 71) | | |
| resistant vanB | | | | |
| vancomycin | vanB-R | gggaaagccacgtcaatacg (SEQ ID NO: 72) | | |
| resistant vanB | | | | |
| vancomycin | vanM-F | gcagagattgccaacaacattga (SEQ ID NO: 73) | | |
| resistant vanM | | | | |
| vancomycin | vanM-R | gcatgttttccaaacgcca (SEQ ID NO: 74) | | |
| resistant vanM | | | | |
| methicillin | mecA-F | aactacggtaacattgatcgcaac (SEQ ID NO: 75) | | |
| resistant mecA | | | | |
| methicillin | mecA-R | gcattcctggaataatgacgcta (SEQ ID NO: 76) | | |
| resistant mecA | | | | |
| methicillin | mecC-F | cggtgaaaatatcccgagtga | | |
| resistant mecC | | (SEQ ID NO: 77) | | |
| methicillin | mecC-R | ggccatatcctgaatctgctaataa | | |
| resistant mecC | | (SEQ ID NO: 78) | | |
| exogenous | IC-F | gcttcttgtggagctcgacaa (SEQ ID NO: 79) | | |
| internal control | | | | |
| exogenous | IC-R | ccgtcagcaacttcgttttca (SEQ ID NO: 80) | | |
| internal control | | | | |
| pseudomonas | TL-P | ccttgacatgctgagaac (SEQ ID NO: 81) | FAM | MGB |
| aeruginosa | | | | |
| escherichia | DC-P | aagggagtaaagttaatacc (SEQ ID NO: 82) | FAM | MGB |
| coli | | | | |
| klebsiella | FK-P | taacctcatcgattgacg (SEQ ID NO: 83) | FAM | MGB |
| pneumoniae | | | | |
| acinetobacter | BM-P | ccttgacatactagaaactt (SEQ ID NO: 84) | FAM | MGB |
| baumannii | | | | |
| staphylococcus | JP-P | tagagccttccccttcg (SEQ ID NO: 85) | VIC | MGB |
| aureus | | | | |
| enterococcus | SC-P | atagagcttccccttcgg (SEQ ID NO: 86) | VIC | MGB |
| faecium | | | | |
| enterococcus | FC-P | agagctttcccttcgg (SEQ ID NO: 87) | VIC | MGB |
| faecalis | | | | |
| enterobacter | YG-P | aaggtgttgtggttaataa (SEQ ID NO: 88) | VIC | MGB |
| cloacae | | | | |
| candida | BS-P | cctgttatcccaayacaaac (SEQ ID NO: 89) | ROX | MGB |
| albicans | | | | |
| candida | GH-P | actaccagcagatgcg (SEQ ID NO: 90) | ROX | MGB |
| glabrata | | | | |
| candida | JPH-P | ttagtatoggaagaactca (SEQ ID NO: 91) | ROX | MGB |
| parapsilosis | | | | |
| candida | RD-P | cagtatgtattctttaacacgttg (SEQ ID NO: 92) | ROX | MGB |
| tropicalis | | | | |
| carbapenem | KPC-P | gccgctgeggctagac (SEQ ID NO: 93) | CY5 | MGB |
| resistant KPC | | | | |
| carbapenem | NDM-P | gccggtcgcgctggc (SEQ ID NO: 94) | CY5 | BHQ2 |
| resistant NDM | | | | |
| carbapenem | OXA48-P | gatttgggcgtggttaagga (SEQ ID NO: 95) | CY5 | MGB |
| resistant | | | | |
| OXA48 | | | | |
| carbapenem | IMP-P | caagagtgatgcgtctc (SEQ ID NO: 96) | CY5 | MGB |
| resistant IMP | | | | |
| vancomycin | vanA-P | cagcagaggagcgagg (SEQ ID NO: 97) | A425 | BHQ1 |
| resistant vanA | | | | |
| vancomycin | vanB-P | agagaatacgaaactcg (SEQ ID NO: 98) | A425 | BHQ1 |
| resistant vanB | | | | |
| vancomycin | vanM-P | tgaaccaatatacatcggaata (SEQ ID NO: 99) | A425 | BHQ1 |
| resistant vanM | | | | |
| methicillin | mecA-P | tgttaaagaagatggtatgtgg (SEQ ID NO: 100) | CY5.5 | BHQ2 |
| resistant mecA | | | | |
| methicillin | mecC-P | ccttttataaagcacaaatc (SEQ ID NO: 101) | CY5.5 | MGB |
| resistant mecC | | | | |
| exogenous | IC-P | cgcgacggatctacgtcacagcg (SEQ ID NO: 102) | CY7 | BHQ3 |
| internal control | | | | |

### EXAMPLE 2

### Digital PCR Amplification System

1. A single amplification system is shown in Table 2

TABLE 2

| | Concentration of | |
|---|---|---|
| Component | storage solution (μM) | Volume (μL) |
| water | | 9.475 |
| 5 × Taq mix | | 3 |
| 50 × positioning dye | | 0.3 |
| F | 400 | 0.0375 |
| R | 400 | 0.0375 |
| P | 100 | 0.0375 |
| IC-F | 400 | 0.0375 |
| IC-R | 400 | 0.0375 |
| IC-P | 100 | 0.0375 |
| IC template | | 1 |
| Bacterium DNA/resistant gene | | 1 |
| total | | 15 |

2. A multiplex amplification system is shown in Table 3

TABLE 3

| | Concentration of | Volume |
|---|---|---|
| Component | storage solution (μM) | (μL/test) |
| water | | 6.4375 |
| 5 × Taq mix | | 3 |
| UP1 | 100 | 0.0375 |
| UP2 | 100 | 0.0375 |
| UP3 | 100 | 0.0375 |
| UP4 | 100 | 0.0375 |
| UP5 | 100 | 0.0375 |
| UP6 | 100 | 0.0375 |
| UP7 | 100 | 0.0375 |
| TL-F | 400 | 0.0375 |
| TL-R | 400 | 0.0375 |
| TL-UP1 | 100 | 0.0375 |
| DC-F | 400 | 0.0375 |
| DC-R | 400 | 0.0375 |
| DC-UP1 | 100 | 0.0375 |
| FK-F | 400 | 0.0375 |
| FK-R | 400 | 0.0375 |
| FK-UP1 | 100 | 0.0375 |
| BM-F | 400 | 0.0375 |
| BM-R | 400 | 0.0375 |
| BM-UP1 | 100 | 0.0375 |
| JP = F | 400 | 0.0375 |
| JP-R | 400 | 0.0375 |
| JP-UP2 | 100 | 0.0375 |
| SC-F | 400 | 0.0375 |
| SC-R | 400 | 0.0375 |
| SC-UP2 | 100 | 0.0375 |
| FC-F | 400 | 0.0375 |
| FC-R | 400 | 0.0375 |
| FC-UP2 | 100 | 0.0375 |
| YG-F | 400 | 0.0375 |
| YG-R | 400 | 0.0375 |
| YG-UP2 | 100 | 0.0375 |
| BS-F | 400 | 0.0375 |
| BS-R | 400 | 0.0375 |
| BS-UP3 | 100 | 0.0375 |
| GH-F | 400 | 0.0375 |
| GH-R | 400 | 0.0375 |
| GH-UP3 | 100 | 0.0375 |
| JPH-F | 400 | 0.0375 |
| JPH-R | 400 | 0.0375 |
| JPH-UP3 | 100 | 0.0375 |
| RD-F | 400 | 0.0375 |
| RD-R | 400 | 0.0375 |
| RD-UP3 | 100 | 0.0375 |
| KPC-F | 400 | 0.0375 |
| KPC-R | 400 | 0.0375 |
| KPC-UP4 | 100 | 0.0375 |
| NDM-F | 400 | 0.0375 |
| NDM-R | 400 | 0.0375 |
| NDM-UP4 | 100 | 0.0375 |
| OXA48-F | 400 | 0.0375 |
| OXA48-R | 400 | 0.0375 |
| OXA48-UP4 | 100 | 0.0375 |
| IMP-F | 400 | 0.0375 |
| IMP-R | 400 | 0.0375 |
| IMP-UP4 | 100 | 0.0375 |
| vanA-F | 400 | 0.0375 |
| vanA-R | 400 | 0.0375 |
| vanA-UP5 | 100 | 0.0375 |
| vanB-F | 400 | 0.0375 |
| vanB-R | 400 | 0.0375 |
| vanB-UP5 | 100 | 0.0375 |
| vanM-F | 400 | 0.0375 |
| vanM-R | 400 | 0.0375 |
| vanM-UP5 | 100 | 0.0375 |
| mecA-F | 400 | 0.0375 |
| mecA-R | 400 | 0.0375 |
| mecA-UP6 | 100 | 0.0375 |
| mecC-F | 400 | 0.0375 |
| mecC-R | 400 | 0.0375 |
| mecC-UP6 | 100 | 0.0375 |
| IC-F | 400 | 0.0375 |
| IC-R | 400 | 0.0375 |
| IC-UP7 | 100 | 0.0375 |
| IC template | | 1 |
| Bacterium DNA/resistant gene | | 1 |
| total | | 15 |

3. An Amplification Program

95° C. 5 min, (95° C. 15 s, 60° C. 1 min)×40

4. A Result Interpretation

(1) A detection result of the single amplification system is shown in Table 4

TABLE 4

| | A425 | FAM | VIC | ROX | CY5 | CY5.5 | CY7 |
|---|---|---|---|---|---|---|---|
| Sample | (copies/μL) | (copies/μL) | (copies/μL) | (copies/μL) | (copies/μL) | (copies/μL) | (copies/μL) |
| pseudomonas | / | 108.44 | / | / | / | / | 258.64 |
| aeruginosa | | | | | | | |
| escherichia coli | / | 218.62 | / | / | / | / | 267.33 |
| klebsiella | / | 58.17 | / | / | / | / | 249.66 |
| pneumoniae | | | | | | | |
| acinetobacter | / | 333.19 | / | / | / | / | 263.55 |
| baumannii | | | | | | | |
| staphylococcus | / | / | 1186.95 | / | / | / | 248.71 |
| aureus | | | | | | | |
| enterococcus | / | / | 2137.44 | / | / | / | 265.89 |
| faecium | | | | | | | |
| enterococcus | / | / | 29.66 | / | / | / | 252.12 |
| faecalis | | | | | | | |
| enterobacter | / | / | 87.11 | / | / | / | 267.81 |
| cloacae | | | | | | | |
| candida albicans | / | / | / | 45.19 | / | / | 257.75 |
| candida glabrata | / | / | / | 86.77 | / | / | 249.46 |
| candida | / | / | / | 83.98 | / | / | 252.33 |
| parapsilosis | | | | | | | |
| candida tropicalis | / | / | / | 36.44 | / | / | 249.6 |
| carbapenem | / | / | / | / | 109.53 | / | 246.28 |
| resistant KPC | | | | | | | |
| carbapenem | / | / | / | / | 218.62 | / | 258.47 |
| resistant NDM | | | | | | | |
| carbapenem | / | / | / | / | 317.4 | / | 245.54 |
| resistant OXA48 | | | | | | | |
| carbapenem | / | / | / | / | 211.09 | / | 247.9 |
| resistant IMP | | | | | | | |
| vancomycin | 319.9 | / | / | / | / | / | 251.11 |
| resistant vanA | | | | | | | |
| vancomycin | 856.71 | / | / | / | / | / | 259.14 |
| resistant vanB | | | | | | | |
| vancomycin | 2365.47 | / | / | / | / | / | 246.77 |
| resistant vanM | | | | | | | |
| methicillin | / | / | / | / | / | 1166.22 | 248.83 |
| resistant mecA | | | | | | | |
| methicillin | / | / | / | / | / | 2312.89 | 264 |
| resistant mecC | | | | | | | |

(2) A detection result of the multiplex amplification system is shown in Table 5

TABLE 5

| | A425 | FAM | VIC | ROX | CY5 | CY5.5 | CY7 |
|---|---|---|---|---|---|---|---|
| Sample | (copies/μL) | (copies/μL) | (copies/μL) | (copies/μL) | (copies/μL) | (copies/μL) | (copies/μL) |
| pseudomonas | 0 | 124.16 | 0 | 0 | 0 | 0 | 248.66 |
| aeruginosa | | | | | | | |
| escherichia coli | 0 | 214.33 | 0 | 0 | 0 | 0 | 243.77 |
| klebsiella | 0 | 55.22 | 0 | 0 | 0 | 0 | 265.56 |
| pneumoniae | | | | | | | |
| acinetobacter | 0 | 324.5 | 0 | 0 | 0 | 0 | 249.35 |
| baumannii | | | | | | | |
| staphylococcus | 0 | 0 | 1150.33 | 0 | 0 | 0 | 243.22 |
| aureus | | | | | | | |
| enterococcus | 0 | 0 | 2140.62 | 0 | 0 | 0 | 249.17 |
| faecium | | | | | | | |
| enterococcus | 0 | 0 | 35.16 | 0 | 0 | 0 | 256.71 |
| faecalis | | | | | | | |
| enterobacter | 0 | 0 | 85.29 | 0 | 0 | 0 | 256.33 |
| cloacae | | | | | | | |
| candida albicans | 0 | 0 | 0 | 48.27 | 0 | 0 | 265.81 |
| candida glabrata | 0 | 0 | 0 | 85.66 | 0 | 0 | 268.66 |
| candida | 0 | 0 | 0 | 89.42 | 0 | 0 | 259.18 |
| parapsilosis | | | | | | | |
| candida tropicalis | 0 | 0 | 0 | 32.15 | 0 | 0 | 246.83 |
| carbapenem | 0 | 0 | 0 | 0 | 106.24 | 0 | 247.5 |
| resistant KPC | | | | | | | |
| carbapenem | 0 | 0 | 0 | 0 | 215.43 | 0 | 258.6 |
| resistant NDM | | | | | | | |
| carbapenem | 0 | 0 | 0 | 0 | 321.99 | 0 | 240.21 |
| resistant OXA48 | | | | | | | |
| carbapenem | 0 | 0 | 0 | 0 | 205.32 | 0 | 253.17 |
| resistant IMP | | | | | | | |
| vancomycin | 314.44 | 0 | 0 | 0 | 0 | 0 | 248.59 |
| resistant vanA | | | | | | | |
| vancomycin | 850.41 | 0 | 0 | 0 | 0 | 0 | 252.33 |
| resistant vanB | | | | | | | |
| vancomycin | 2377.82 | 0 | 0 | 0 | 0 | 0 | 242.59 |
| resistant vanM | | | | | | | |
| methicillin | 0 | 0 | 0 | 0 | 0 | 1145.54 | 262.13 |
| resistant mecA | | | | | | | |
| methicillin | 0 | 0 | 0 | 0 | 0 | 2320.17 | 247.7 |
| resistant mecC | | | | | | | |

The results showed that the Pseudomonas aeruginosa, Escherichia coli, Klebsiella pneumoniae and Acinetobacter baumannii only had detection values in FAM and CY7, and there was no detection value in other channels.

The Staphylococcus aureus, Enterococcus faecium, Enterococcus faecalis and Enterobacter cloacae only had detection values in VIC and CY7, and there was no detection value in other channels.

The Candida albicans, Candida glabrata, Candida parapsilosis and Candida tropicalis only had detection values in ROX and CY7, and there was no detection value in other channels.

The carbapenem resistant genes only had detection values in CY5 and CY7, and there was no detection value in other channels.

The vancomycin resistant genes only had detection values in A425 and CY7, and there was no detection value in other channels.

The methicillin resistant genes only had detection values in CY5.5 and CY7, and there was no detection value in other channels.

In summary, the primers and probes provided by the present application can accurately detect a simulated sample, and the detection results were fully consistent with an expectation, which indicated that an accuracy of the detection system was normal. The system for detecting a plurality of pathogenic microorganisms and resistant genes in the present application had specificity. The detection results of the two detection systems were basically consistent, which indicated that the multiplex detection system of the present application has stability.

The above are the preferred embodiments of the present application. It should be noted that, those skilled in the art can make several changes or modifications according without departing from the principles of the present application, which should be covered within the protection scope of the present application.

What is claimed is:

1. A universal probe, wherein, from 5′ end to 3′ end, the universal probe sequentially comprises: quenching group-fragment A-fluorophore-fragment B-C3;
the fragment A has a nucleotide sequence selected from a group consisting of SEQ ID NO:1-7; and
the fragment B has a nucleotide sequence selected from a group consisting of SEQ ID NO:8-14.

2. The universal probe according to claim 1, wherein, from 5′ end to 3′ end, the universal probe sequentially comprises one selected from a group consisting of:
quenching group-fragment A having a nucleotide sequence of SEQ ID NO:1-fluorophore-fragment B-C3 having a nucleotide sequence of SEQ ID NO:8;
quenching group-fragment A having a nucleotide sequence of SEQ ID NO:2-fluorophore-fragment B-C3 having a nucleotide sequence of SEQ ID NO:9;
quenching group-fragment A having a nucleotide sequence of SEQ ID NO:3-fluorophore-fragment B-C3 having a nucleotide sequence of SEQ ID NO:10;
quenching group-fragment A having a nucleotide sequence of SEQ ID NO:4-fluorophore-fragment B-C3 having a nucleotide sequence of SEQ ID NO:11;
quenching group-fragment A having a nucleotide sequence of SEQ ID NO:5-fluorophore-fragment B-C3 having a nucleotide sequence of SEQ ID NO:12;
quenching group-fragment A having a nucleotide sequence of SEQ ID NO:6-fluorophore-fragment B-C3 having a nucleotide sequence of SEQ ID NO:13; and
quenching group-fragment A having a nucleotide sequence of SEQ ID NO:7-fluorophore-fragment B-C3 having a nucleotide sequence of SEQ ID NO:14.

3. The universal probe according to claim 1, wherein, the fluorophore is selected from a group consisting of FAM, VIC, ROX, CY5, A425, CY5.5 and CY7; and the quenching group is selected from a group consisting of BHQ1, BHQ2 and BHQ3.

4. A primer-probe set for detecting one selected from a group consisting of a pathogenic microorganism and a resistant gene, comprising the universal probe according to claim 1, a specific primer pair and a single-modified fluorescence-free probe, wherein the specific primer pair targets the pathogenic microorganism and the resistant gene.

5. The primer-probe set according to claim 4, wherein, a nucleotide sequence of the single-modified fluorescence-free probe is reversely complementary to the nucleotide sequence of the fragment B in the universal probe.

6. The primer-probe set according to claim 5, wherein, 3′ end of the single-modified fluorescence-free probe is labeled with the quenching group.

7. The primer-probe set according to claim 6, wherein, the quenching group labeled at 3′ end of the single-modified fluorescence-free probe is selected from MGB, BHQ1, BHQ2 and BHQ3.

8. The primer-probe set according to claim 4, wherein, the single-modified fluorescence-free probe has a nucleotide sequence selected from a group consisting of SEQ ID NO:15-36.

9. The primer-probe set according to claim 4, wherein, the pathogenic microorganism comprises one selected from a group consisting of Pseudomonas aeruginosa, Escherichia coli, Klebsiella pneumoniae, Acinetobacter baumannii, Staphylococcus aureus, Enterococcus faecium, Enterococcus faecalis, Enterobacter cloacae, Candida albicans, Candida glabrata, Candida parapsilosis and Candida tropicalis; and
the resistant gene comprises one selected from a group consisting of carbapenem resistant KPC gene, carbapenem resistant NDM gene, carbapenem resistant OXA48 gene, carbapenem resistant IMP gene, vancomycin resistant vanA gene, vancomycin resistant vanB gene, vancomycin resistant vanM gene, methicillin resistant mecA gene and methicillin resistant mecC gene.

10. The primer-probe set according to claim 9, wherein, a primer pair targeting the Pseudomonas aeruginosa has a nucleotide sequence selected from a group consisting of SEQ ID NO:37-38;
a primer pair targeting the Escherichia coli has a nucleotide sequence selected from a group consisting of SEQ ID NO:39-40;
a primer pair targeting the Klebsiella pneumoniae has a nucleotide sequence selected from a group consisting of SEQ ID NO:41-42;
a primer pair targeting the Acinetobacter baumannii has a nucleotide sequence selected from a group consisting of SEQ ID NO:43-44;
a primer pair targeting the Staphylococcus aureus has a nucleotide sequence selected from a group consisting of SEQ ID NO:45-46;
a primer pair targeting the Enterococcus faecium has a nucleotide sequence selected from a group consisting of SEQ ID NO:47-48;
a primer pair targeting the Enterococcus faecalis has a nucleotide sequence selected from a group consisting of SEQ ID NO:49-50;
a primer pair targeting the Enterobacter cloacae has a nucleotide sequence selected from a group consisting of SEQ ID NO:51-52;
a primer pair targeting the Candida albicans has a nucleotide sequence selected from a group consisting of SEQ ID NO:53-54;
a primer pair targeting the Candida glabrata has a nucleotide sequence selected from a group consisting of SEQ ID NO:55-56;
a primer pair targeting the Candida parapsilosis has a nucleotide sequence selected from a group consisting of SEQ ID NO:57-58;
a primer pair targeting the Candida tropicalis has a nucleotide sequence selected from a group consisting of SEQ ID NO:59-60;
a primer pair targeting the carbapenem resistant KPC gene has a nucleotide sequence selected from a group consisting of SEQ ID NO:61-62;
a primer pair targeting the carbapenem resistant NDM gene has a nucleotide sequence selected from a group consisting of SEQ ID NO:63-64;
a primer pair targeting the carbapenem resistant OXA48 gene has a nucleotide sequence selected from a group consisting of SEQ ID NO:65-66;
a primer pair targeting the carbapenem resistant IMP gene has a nucleotide sequence selected from a group consisting of SEQ ID NO:67-68;
a primer pair targeting the vancomycin resistant vanA gene has a nucleotide sequence selected from a group consisting of SEQ ID NO:69-70;
a primer pair targeting the vancomycin resistant vanB gene has a nucleotide sequence selected from a group consisting of SEQ ID NO:71-72;
a primer pair targeting the vancomycin resistant vanM gene has a nucleotide sequence selected from a group consisting of SEQ ID NO:73-74;
a primer pair targeting the methicillin resistant mecA gene has a nucleotide sequence selected from a group consisting of SEQ ID NO:75-76; and, a primer pair targeting the methicillin resistant mecC gene has a nucleotide sequence selected from a group consisting of SEQ ID NO:77-78.

11. A kit, comprising one selected from a group consisting of the universal probe described in claim 1 or a primer-probe set for detecting one selected from a group consisting of a pathogenic microorganism and a resistant gene, and an auxiliary agent or a carrier, wherein the primer-probe set for detecting one selected from a group consisting of a pathogenic microorganism and a resistant gene comprises the universal probe according to claim 1, a specific primer pair and a single-modified fluorescence-free probe, and the specific primer pair targets the pathogenic microorganism and the resistant gene.
